(19) United States
(12) Patent Application Publication
(10) Pub. No.: US 2026/0144661 A1
(43) Pub. Date: May 28, 2026

(54) UNIVERSAL ANKLE-FOOT ORTHOSIS

(71) Applicant: The Regents of the University of Michigan, Ann Arbor, MI (US)

(72) Inventors: Nikko Van Crey, Ann Arbor, MI (US); Elliott J. Rouse, Ann Arbor, MI (US)

(21) Appl. No.: 19/122,135

(22) PCT Filed: Oct. 17, 2023
(86) PCT No.: PCT/US2023/077063

§ 371 Date: Apr. 17, 2025

Related U.S. Application Data
(60) Provisional application No. 63/416,910, filed on Oct. 17, 2022.

(51) Int. Cl. A61F 5/01

(52) U.S. Cl. CPC A61F 5/0127; A61F 2005/0151; A61F 2005/0158; A61F 2005/0179

(57) ABSTRACT

An ankle-foot orthosis includes a leg portion and a foot portion. The foot portion is biased toward an equilibrium angle with respect to the leg portion, and the equilibrium angle is adjustable to accommodate different users'natural stance. An adjustable leg stay permits fitting the orthosis to multiple different users, and the orthosis can be fitted to either leg of a user with only a foot plate change. A variable stiffness spring and cam system modulate ankle joint stiffness according to a pre-defined cam profile. The contact point of the cam system is forward of the user's heel and thus closer to the ankle joint, enabling reduced-size components. Two rotational degrees of freedom are provided in plan-tarflexion/dorsiflexion and inversion/eversion.

### TECHNICAL FIELD

The present disclosure relates generally to an orthotic device and related methods and, more particularly, to ankle-foot orthoses.

### BACKGROUND

Difficulty walking and climbing stairs is the most prevalent disability in the United States and is often attributed to the ankle joint, which plays a critical role in balance and provides most of the net-positive mechanical work during level ground walking. Without proper ankle function, a person may have trouble maintaining ground clearance (i.e., drop foot) during leg swing phase, supporting body weight during midstance, and/or propelling the body forward during terminal stance phase. Ankle-foot orthoses (AFOs) may be used to address these problems. While some AFOs can improve locomotive function, their drawbacks may include restricted range-of-motion, reduced ankle push-off power, hysteresis, and fixed ankle mechanics.

AFOs generally fall within two classes: passive mechanisms that act as springs in parallel with the body, and powered mechanisms (i.e., exoskeletons) that can perform net-positive mechanical work during the gait cycle. Most commercially available ankle-foot orthoses are passive, compliant plastic structures that span the ankle joint to help ankle stability and mitigate gait abnormalities. Such passive devices have fixed mechanics typically configured for walking on level ground and cannot modulate stiffness to assist with navigating stairs or ramps, for example. Moreover, passive orthoses are typically provided in a one-size-fits-all configuration or include features that must be custom molded to a particular individual's limb to provide proper function. Some AFOs permit changes in stiffness through disassembly and reassembly, but none can change stiffness while walking or replicate a biomimetic torque-angle relationship. While ankle exoskeletons have shown some promise as an alternative to passive AFOs, those devices face challenges with reliability, weight, and cost, which has hindered their commercialization and widespread use.

### SUMMARY

An embodiment of an ankle-foot orthosis includes a leg portion and a foot portion. The foot portion is biased toward an equilibrium angle with respect to the leg portion, and the equilibrium angle is adjustable.

In various embodiments, the orthosis includes a spring that biases the foot portion toward the equilibrium angle, and the spring has a variable stiffness.

In various embodiments, the orthosis includes a cam and a cam follower, one of which is mounted for movement with the leg portion about a pivot joint coupling the leg portion to the foot portion and the other of which is mounted for movement with the foot portion about the pivot joint. The cam and the cam follower are biased against each other when the foot portion is away from the equilibrium angle.

In various embodiments, the orthosis includes a pivot joint coupling the leg portion to the foot portion, and the pivot joint provides two rotational degrees of freedom.

In various embodiments, the orthosis includes a pivot joint coupling the leg portion to the foot portion, and the equilibrium angle is adjustable about the pivot joint in dorsiflexion and plantarflexion directions.

In various embodiments, the leg portion includes a frame and a leg stay. The frame has a first end coupled with the foot portion at a pivot joint and a second end coupled with the leg stay. A position of the second end of the frame relative to the leg stay is adjustable about the pivot joint. In various embodiments, the leg portion includes a coupling having a first pivot arm and a second pivot arm. The first pivot arm has a first end coupled with a leg stay and a second end coupled with the second pivot arm at an adjustable joint. The second pivot arm has a first end coupled with a leg portion frame and a second end at the adjustable joint. The adjustable joint provides linear adjustment of the second pivot arm relative to the first pivot arm to move the frame about the pivot joint. A position of the first end of the second pivot arm may be linearly adjustable relative to the frame. The coupling may provide pivoting movement of the frame relative to the leg stay about two parallel axes.

An embodiment of the ankle-foot orthosis includes a leg portion and a foot portion. The foot portion has an equilibrium angle with respect to the leg portion, and a biasing force is applied to the foot portion anterior to the heel of a person wearing the orthosis when the foot portion is away from the equilibrium angle.

In various embodiments, the equilibrium angle is adjustable.

In various embodiments, the orthosis includes a pivot joint coupling the leg portion to the foot portion. The pivot joint provides two rotational degrees of freedom including a first degree of freedom about a first axis in dorsiflexion and plantarflexion directions, and a second degree of freedom about a second axis in inversion and eversion directions.

In various embodiments, the leg portion includes a frame and a leg stay. The frame has a first end coupled with the foot portion at a pivot joint and a second end coupled with the leg stay. The frame is lateral to the leg of the person wearing the orthosis.

In various embodiments, the orthosis includes a cam and a cam follower, one of which is mounted for movement with the leg portion about a pivot joint coupling the leg portion to the foot portion and the other of which is mounted for movement with the foot portion about the pivot joint. The cam and the cam follower are biased against each other by a spring when the foot portion is away from the equilibrium angle. A torque about the pivot joint resulting from a biasing force of the spring is a function of the angle of the foot portion with respect to the leg portion. The function is at least partially defined by a profile of the cam and by a stiffness of the spring. The stiffness of the spring may be variable.

In various embodiments, the leg portion comprises a spring, a spring support, a frame, and a leg stay. The frame has a first end coupled with the foot portion at a pivot joint and a second end coupled with the leg stay. The spring is a cantilever having one end attached to the frame and an opposite free end that applies the biasing force. The spring support is a simple support for the cantilever and is movably mounted on the frame for movement between the ends of the cantilever to vary the stiffness of the spring. Movement of the spring support may be motorized, and the spring support may be posterior to the spring.

In various embodiments, a cam is mounted for movement with the foot portion and a cam follower is attached to a spring such that the cam pivots about an axis of a pivot joint and the cam follower is biased against the cam by the spring. The cam may be at the pivot joint.

An embodiment of the ankle-foot orthosis includes a leg portion and a foot portion coupled at a pivot joint having two rotational degrees of freedom.

In various embodiments, the two degrees of freedom include a first degree of freedom about a first axis in dorsiflexion and plantarflexion directions, and a second degree of freedom about a second axis in inversion and eversion directions.

In various embodiments, the pivot joint comprises a joint body. The joint body and the foot portion pivot together about a first axis with respect to the leg portion, and the foot portion pivots about a second axis with respect to the joint body.

In various embodiments, the orthosis includes a sensor configured to determine an angle of the foot portion with respect to the leg portion about a first axis.

In various embodiments, a range of angular motion of the foot portion with respect to the leg portion about first and second axes is adjustable.

An embodiment of the ankle-foot orthosis is configured for use on one of: a right leg or a left leg. The orthosis is convertible for use on the other leg by replacing only a foot plate of the orthosis.

In various embodiments, the orthosis includes a pivot arm, a spring assembly, and a leg stay. The pivot arm is configured for removable attachment to the foot plate at one end and forms part of a pivot joint at an opposite end. The spring assembly forms part of the pivot joint at one end and is removably attached to a coupling at an opposite end. The leg stay is attached to the coupling. When converted for use on the other leg, an orientation of the pivot arm, the pivot joint, and the spring assembly does not change, and the coupling is rotated 180 degrees about an axis extending in the anterior and posterior directions. The orthosis may include an angle sensor mounted on a first side of the pivot joint, and the sensor may be mounted on an opposite second side of the pivot joint when converted for use on the other leg.

An embodiment of a method of fitting a ankle foot orthosis to a person includes the step of adjusting an equilibrium angle of the orthosis to match a stance angle of the person.

In various embodiments, the orthosis includes a cam having a cam profile, a cam follower, and a spring assembly configured to bias the cam and cam follower toward each other when the orthosis is not at the equilibrium angle. The method includes: (a) unlocking an adjustable joint coupling the spring assembly with a leg stay of the orthosis; (b) rotating the spring assembly about a pivot joint of the orthosis until the cam follower is at an equilibrium point along the cam profile; and (c) locking the adjustable joint after step (b). Steps (b) and (c) are performed while the person is wearing the orthosis and in a standing position.

In various embodiments, the adjustable joint is a linearly adjustable joint.

In various embodiments, the method includes the step of adjusting a reaction force applied to the leg of the person wearing the orthosis during dorsiflexion and plantarflexion.

In various embodiments, the method includes: (a) unlocking an adjustable joint coupling a spring assembly with a leg stay of the orthosis; (b) vertically adjusting the leg stay relative to the spring assembly; and (c) locking the adjustable joint after step (b).

An embodiment of the ankle-foot orthosis includes a leg portion and a foot portion biased toward an equilibrium angle about a pivot joint, and the pivot joint has a non-linear torque-angle function.

In various embodiments, the orthosis includes a spring that biases the foot portion toward the equilibrium angle, and the spring has a variable stiffness.

In various embodiments, the orthosis includes a cam and a cam follower, one of which is mounted for movement with the leg portion about a pivot joint coupling the leg portion to the foot portion and the other of which is mounted for movement with the foot portion about the pivot joint. The cam and the cam follower are biased against each other when the foot portion is away from the equilibrium angle, and the non-linear torque-angle function is defined at least in part by a cam profile of the cam.

In various embodiments, the equilibrium angle is adjustable.

In various embodiments, the pivot joint has two rotational degrees of freedom.

It is contemplated that any one or more of the above listed features, any one or more of the features in the appended figures, and/or any one or more of the features in the following description of embodiments can be combined in any technically feasible combination to define at least a portion of a claimed invention.

### BRIEF DESCRIPTION OF THE DRAWINGS

FIG. 1 is a perspective view of an example of a foot-ankle orthosis;

FIG. 2 is a side elevation and partial cutaway view of an example of a foot-ankle orthosis;

FIG. 3 is an enlarged view of a portion of FIG. 2;

FIG. 4 is an example of a torque-angle function of an embodiment of a foot-ankle orthosis;

FIG. 5 is an exploded view of a leg stay and coupling of the orthosis of FIG. 1;

FIG. 6 is a perspective view of a lower portion of the orthosis of FIG. 1 with the pivot joint in exploded view;

FIG. 7 is a perspective view of a removable and replaceable cam of the orthosis;

FIG. 8 is a perspective view of a removable and replaceable spring of the orthosis;

FIG. 9 is a perspective view of a foot portion of the orthosis integrated into a shoe of an intended user; and

FIG. 10 is a chart of torque-angle functions of a prototype orthosis overlaid on model-predicted torque-angle functions.

### DESCRIPTION OF EMBODIMENTS

Described below is a universal ankle-foot orthosis that addresses many of the deficiencies of previous AFOs. The orthosis may be constructed with a customized and interchangeable torque-angle relationship, variable joint stiffness, adjustability for proper fit and function on multiple different people, multiple controlled degrees of freedom about the ankle joint, and major components that are non-specific to the right or left leg. The orthosis may be quasi-passive, meaning that it may include a powered actuator providing relative motion among individual elements of the device in a manner that does not perform net-positive work during the gait cycle.

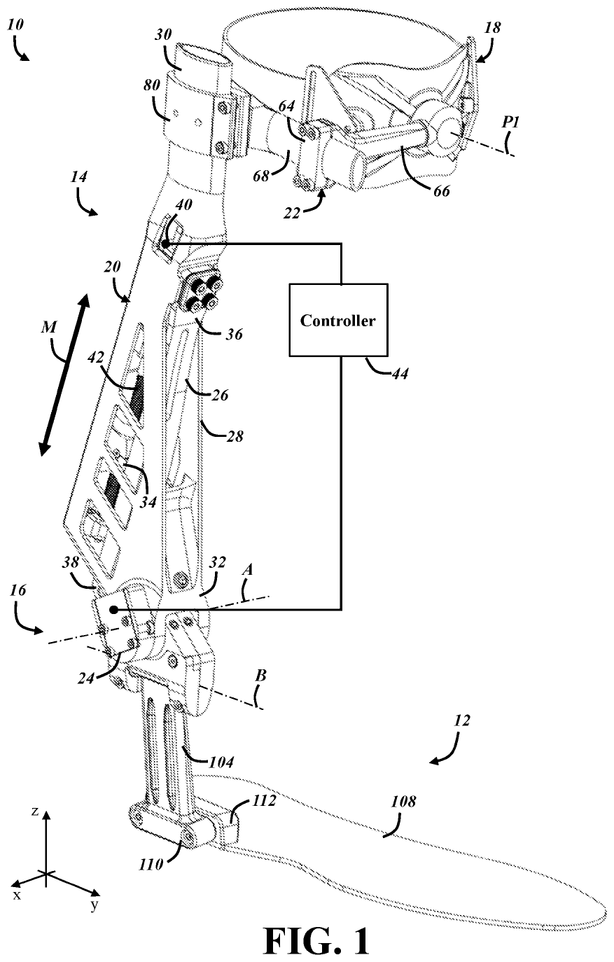

FIG. 1 is a perspective view of an example of a foot-ankle orthosis 10 embodying one or more of these features. The illustrated orthosis 10 includes a foot portion 12, a leg portion 14, and a joint 16 that permits pivotal movement of the foot and leg portions relative to one another. The foot portion 12 is configured to move with the foot of a person wearing the orthosis 10. The leg portion 14 is configured to move with the leg-specifically, the lower portion of the leg between the knee and the ankle-of a person wearing the orthosis 10. The joint 16 is a pivot joint that couples the foot portion 12 and the leg portion 14 in a manner that permits their relative movement in directions that emulate movement of a human ankle. The primary direction of permitted relative movement is rotation about a horizontal first pivot axis A, which is analogous to dorsiflexion and plantarflexion of the foot about the ankle. A secondary direction of permitted relative movement is rotation about a second horizontal pivot axis B, perpendicular to the first axis A. The secondary direction is analogous to inversion and eversion of the foot about the ankle. Other directions of permitted movement are not necessarily excluded, such as rotational movement about a third axis that is analogous to adduction and abduction of the foot about the ankle. Linear movements may also be accommodated.

The illustrated leg portion 14 includes a leg stay 18, a spring assembly 20, a coupling 22, and an angle sensor 24. The leg stay 18 is configured for removable attachment of the leg portion 14 to the lower leg at a fixed location between the knee and the ankle of a person wearing the orthosis 10. The spring assembly 20 includes a spring 26 carried by a rigid frame 28 and is configured to apply a biasing force and a resulting restorative torque to the foot portion 12 when the foot portion is away from an equilibrium angle with respect to the leg portion 14. A first (upper) end 30 of the frame 28 is coupled with the leg stay 18 via the coupling 22, and a second (lower) end 32 of the frame is coupled with the foot portion 12 via the joint 16.

The spring 26 is configured to temporarily store mechanical energy during forced rotation of the foot portion 12 about the first axis A of the joint 16 away from the equilibrium angle. Energy stored in the spring 26 applies a restorative torque about the first axis A of the joint 16 in a direction toward the equilibrium angle. The frame 28 provides an effective lever arm applying a resultant force to the leg of the person wearing the orthosis 10 based at least in part on the magnitude of the restorative torque and the distance between the first axis A of the joint 16 and the leg stay 18.

The spring 26 may have an adjustable or variable stiffness. In the illustrated example, the spring assembly 20 includes a moveable support 34 carried by the frame 28. The spring 26 of FIG. 1 is a cantilever having a first end 36 rigidly mounted to the frame 28 and a second or opposite free end 38. The support 34 is a simple support for the cantilever and supports the cantilever at a location between its first and second ends 36, 38. The effective stiffness of the spring 26 is a function of the location of the moveable support 34 along the length of the spring—i.e., movement of the support 34 along direction M away from the first end 36 increases the effective stiffness of the spring, while movement of the support 34 toward the first end 36 decreases the effective stiffness of the spring. An interface 35 of the frame 28 and moveable support 34 along which the support moves may have low friction surfaces (e.g., PTFE, acetal, UHMWPE, etc.) at one or both sides of the interface.

The illustrated spring 26 has a variable stiffness, meaning that the stiffness of the spring can be actively changed during use of the orthosis 10, such as during a gait cycle. As used here, non-linear springs having a stiffness that passively varies with deflection are not considered variable stiffness springs on their own, although the spring 26 may also be a non-linear spring. The spring 26 may for example have one stiffness or range of stiffnesses during terminal gate phase and a different stiffness or range of stiffnesses during subsequent heel strike via movement of the support 34 along the spring 26 during leg swing. The spring assembly 20 may include an actuator 40 and a transmission 42 that converts actuator motion to movement of the support 34. In the example of FIG. 1, the actuator 40 is an electric motor and the transmission 42 is a lead screw. The frame 28 slidably supports the support 34, and the lead screw 42 converts rotational motion of the motor shaft to linear motion of the support 34 along the frame. Alternatively, the spring 26 may have an adjustable stiffness, meaning that the stiffness can be adjusted to a higher or lower value while the orthosis is not being worn or while being worn with the user standing still. A similar motorized system or a manual adjuster may be used to move the support 34 to a fixed location along the spring 26 during adjustment.

The orthosis 10 may include an on-board control system comprising a controller 44 that receives information from one or more sensors and controls the stiffness of the variable stiffness spring 26 based at least partly on the received information. Each sensor obtains information pertinent to the function of the orthosis 10 such as position, direction, and/or speed of movement one element relative to another, a force or force distribution along an element of the orthosis, environmental conditions, etc. One such sensor is the angle sensor 24, which may be an encoder that provides an angle of the foot portion 12 relative to the leg portion 14 and/or relative to the equilibrium angle of the orthosis 10. The controller 44 may be configured, for example, to move the spring support 34 only when the orthosis 10 is at its equilibrium angle, when resistance to movement of the support is minimized. Another encoder associated with the actuator 40 and/or transmission 42 can provide the controller with information related to the instant position of the spring support 34 along the spring 26. The controller 44 may for example receive information from sensors related to the relative position of the orthosis from stride-to-stride to thereby determine whether the user has begun to ascend or descend stairs or a ramp and then modify the torque-angle character of the orthosis 10 in real-time to accommodate the accompanying change in gait. The orthosis 10 may also include a power source (not shown) such as one or more batteries to power the actuator 40, controller 44, angle sensor 24, and any other powered feature.

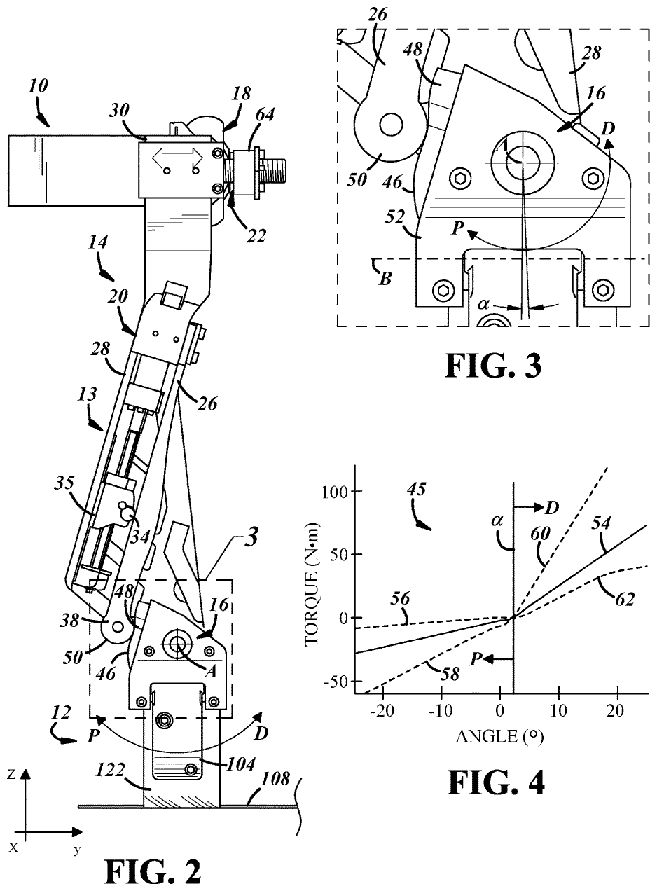

FIG. 2 is a side elevation and partial cutaway view of an embodiment of the orthosis 10 similar to that of FIG. 1, FIG. 3 is an enlarged view of a portion of FIG. 2, and FIG. 4 is an exemplary torque-angle function 45. With reference to FIGS. 2-4, the torque-angle function 45 of the orthosis 10 may be at least partly defined by a cam profile 46. The illustrated orthosis 10 includes a cam 48 and a cam follower 50, one of which is mounted for movement with the foot portion 12 about the first axis A of the pivot joint 16 and the other of which is mounted for movement with the leg portion 14 about the same axis. In this case, the cam 48 is mounted on a body 52 of the pivot joint 16, and the cam follower 50 is mounted on the free end 38 of the spring 26. The cam 48 and cam follower 50 may be reversed in some embodiments with the cam profile 44 provided by a surface of the spring 26 or by a cam mounted on the spring, for example. The illustrated cam follower 48 is a roller with its associated axle affixed to the spring 26.

The torque-angle function of FIG. 4 is associated with a quasi-passive orthosis 10 equipped with a variable stiffness spring 26, cam 48, and cam follower 50 as in the examples of FIGS. 1-3. The chart illustrates restorative torque about the first axis A of the pivot joint 16 as a function of an angle defined between the foot portion 12 and leg portion 14 of the orthosis 10. The solid line is a primary curve 54 representing the torque-angle relationship at a nominal position of the spring support 34 along the spring 26—i.e., torque vs. angle with a constant spring stiffness in the example of FIG. 4. The region between boundary 56 and boundary 58 is a range of available restorative torques when the foot portion 12 is in plantarflexion P relative to an equilibrium angle a, and the region between boundary 60 and boundary 62 is a range of available restorative torques when the foot portion in in dorsiflexion D relative to the equilibrium angle. The equilibrium angle a is a characteristic angle of the orthosis 10, or of the foot portion 12 with respect to the leg portion 14, at which the restorative torque is zero or where the spring biasing force is at a local minimum. In this example, the equilibrium angle a is approximately 2 degrees in the dorsiflexion direction with respect to a vertical reference angle.

Stated in terms of the examples in the figures, FIGS. 2 and 3 are lateral or outboard views of an orthosis 10 configured for use on the right leg of a user in which the orthosis is illustrated in plantarflexion by approximately 2 degrees with respect to the equilibrium angle a. In the illustrated state and view, and with the chart of FIG. 4 governing the torque-angle relationship, a restorative torque would rotate the leg portion 14 about axis A by approximately 2 degrees clockwise with the foot portion 12 held stationary and the leg portion 14 free to move.

The shape of the primary curve 54 of FIG. 4 is defined primarily by the shape of the cam profile 46, while its magnitude is defined in part by the nominal stiffness of the spring 26 and the length of the moment arm of the spring biasing force about the axis A. Each boundary 56-62 is defined primarily by the minimum and maximum available stiffnesses of the variable stiffness spring 26. Boundary 56 represents the torque-angle relationship when the foot portion 12 is in plantarflexion and the spring 26 is at its minimum stiffness—i.e., when the spring support 34 is at its furthest from the cam follower 50. Boundary 58 represents the torque-angle relationship when the foot portion 12 is in plantarflexion and the spring 26 is at its maximum stiffness—i.e., when the spring support 34 is at its closest to the cam follower 50. Boundary 60 represents the torque-angle relationship when the foot portion 12 is in dorsiflexion and the spring 26 is at its maximum stiffness, and boundary 62 represents the torque-angle relationship when the foot portion 12 is in dorsiflexion and the spring 26 is at its minimum stiffness. With a control system as described above, the controller 44 thus has the entire range of restorative torques between the boundaries 56, 58 and 60, 62 to choose from and can be programmed to vary the stiffness of the spring 26 to achieve the desired restorative torque at any degree of plantarflexion or dorsiflexion.

The slope of the torque-angle function represents the stiffness of the joint 16 as an effective torsional spring. In the example of FIG. 4, the torque-angle function at a constant stiffness of the spring 26 is linear, with the joint 16 having a first stiffness in plantarflexion (P) and a greater second stiffness in dorsiflexion (D). This is by design of the cam profile 46. In other examples, the torque-angle function can be made non-linear via the cam profile 46 such that effective joint stiffness is passively varied as a function of joint angle, even without varying the stiffness of the spring. It is thus possible that the orthosis 10 has a variable joint stiffness about the pivot axis A even without the illustrated variable stiffness spring 26 and spring assembly 20.

The illustrated spring and cam configuration offers several advantages over prior art orthoses. For example, the biasing force responsible for the restorative torque is applied relatively close to the joint 16 and about a defined axis A. While compliant plastic orthoses possess a very complex torque-angle relationship rivaling the complexity of a human joint, it is usually not by design. Complexity in that relationship can be undesirable in terms of predictability, with the instantaneous location of the rotational axis continuously changing and rotational motion being uncontrolled about other axes. In some prior art orthoses, a major portion or essentially all of the biasing force is applied posterior to the Achilles tendon and/or the heel of the person wearing the orthosis. By locating the cam profile 46 anterior to or forward of the Achilles tendon and the heel of the person wearing the illustrated orthosis 10, the size of the cam 48 can be significantly smaller than if it was located posterior to the heel or Achilles tendon. For example, to achieve the primary curve of FIG. 4 with a cam located along the user's Achilles tendon, the cam profile 46 would have to be lengthened due to the arcuate movement of the cam follower for a given change in angle being increased with increased distance from the joint 16. The relatively small cam 48 eliminates extraneous material and makes the orthosis 10 less massive.

The relatively short distance between the cam profile 46 and the joint axis A is made possible by locating the joint 16, spring assembly 20, and cam 48 lateral to or medial to the leg of the person wearing the orthosis 10. In the illustrated examples, those components are lateral to or outboard of the leg of the user. They are also lateral to the leg stay 18 and to a sagittal plane bisecting the leg stay and extending through the foot portion 12. Another feature minimizing the distance between the cam profile 46 and the joint axis A is the location of the spring support 34, actuator 40, and transmission 42 posterior to or rearward of the spring 26. While it is possible to locate at least the actuator 40 and transmission 42 anterior to or forward of the spring 26, packaging constraints could then force the cam profile 46 further from the pivot axis A.

In some embodiments, the equilibrium angle a of the orthosis 10 is adjustable. In particular, the angle between the foot portion 12 and the leg portion 14 is adjustable so that the restorative torque is zero or minimized when a person wearing the orthosis is standing in their natural stance. For instance, a person wearing the orthosis 10 may have a natural standing stance in which their foot is farther in dorsiflexion or plantarflexion than the foot portion 12 of the orthosis is at its equilibrium angle. The orthosis 10 may be adjustable about the axis A of the pivot joint 16 to effectively change the equilibrium angle of the orthosis to match the user's stance angle by moving the frame 28 and spring assembly 20 about the axis A.

To accommodate this adjustment, the coupling 22 by which the spring assembly 20 is attached to the leg stay 18 may include an adjustable joint 64, examples of which are illustrated in FIGS. 1 and 2. Each of the illustrated adjustable joints 64 is linearly adjustable to move the end 30 of the frame 28 and spring assembly 20 toward or away from a laterally extending arm 66 of the coupling 22. In the example of FIG. 2, the adjustable joint 64 is a threaded joint in which a threaded rod is rotated within the joint to push the end 30 of the frame 28 away from or pull the end of the frame toward the joint to thereby change the equilibrium angle of the orthosis 10. The adjustable joint 64 of FIG. 1 is a lockable and unlockable clamp joint that permits linear adjustment of another arm 68 of the coupling to move the end 30 of the frame 28 toward or away from the laterally extending arm 66.

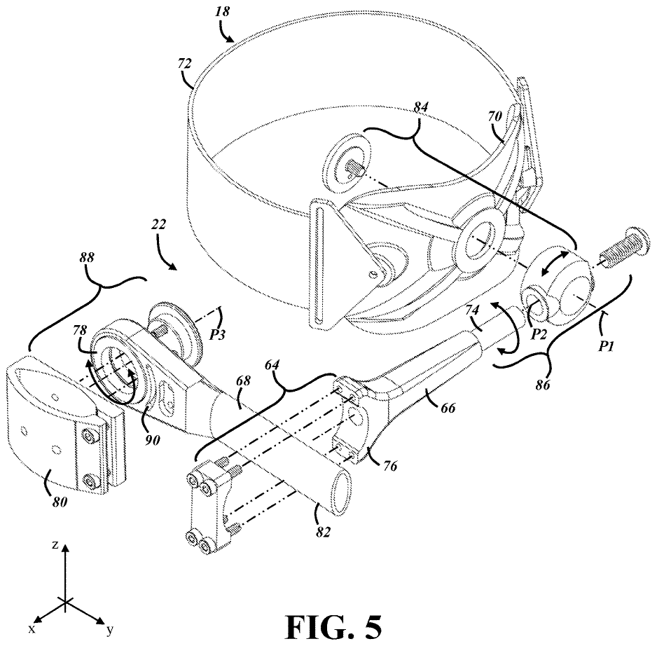

FIG. 5 is an exploded view of the leg stay 18 and coupling 22 of FIG. 1. The leg stay 18 includes a plate 70 and a strap 72 that together encircle the leg of the person wearing the orthosis 10. The plate 70 is a shin plate with a contoured or conformal inner surface intended to be affixed to the anterior or front side of the lower leg below the knee via tension in the strap 72 (e.g., a boa strap). The strap 72 is adjustable to accommodate multiple leg sizes.

The illustrated coupling 22 includes two pivot arms and at least a portion of five joints. The laterally extending arm 66 is a first pivot arm and the other arm 68 is a second pivot arm. The first pivot arm 66 has a first end 74 coupled with the leg stay 18 and a second end 76 coupled with the second pivot arm 68 at a first adjustable joint 64. The second pivot arm 68 has a first end 78 coupled with the frame 28 of the spring assembly 20 (see FIG. 1) at a second adjustable joint 80 and a second end 82 coupled with the first pivot arm 66 at the first adjustable joint 64. Each of the adjustable joints 64, 80 is linearly adjustable and each adjustable joint is a lockable and unlockable clamp joint in the illustrated example.

The first joint 64 permits a single linear degree of freedom between the first and second pivot arms 66, 68 in forward and rearward directions when unlocked and is a rigid joint when locked. As noted above, the first adjustable joint 64 permits adjustment of the equilibrium angle of the orthosis via forward or rearward movement of the top end 30 of the spring assembly 20. The second joint 80 permits a single linear degree of freedom between the second pivot arms 68 and the end 30 of the frame 28 in a vertical direction when unlocked and is a rigid joint when locked. The second adjustable joint 80 permits vertical adjustment of the leg stay 18 along the leg of the user, which changes the effective lever arm with which the reaction force of the spring 26 is applied to the user's leg.

Because the spring assembly 20 pivots about the first axis A of the pivot joint 16 when the equilibrium angle is adjusted, movement of the top end 30 of the spring assembly 20 is arcuate when moved horizontally via the adjustable joint 64. To accommodate that arcuate movement, the coupling 22 includes one or more pivot joints. The illustrated example includes three pivot joints 84-88. The first pivot arm 66 is coupled with the plate 70 of the leg stay 18 via a first pivot joint 84 for rotation about a first pivot axis PI extending in forward and rearward directions, and via a second pivot joint 86 for rotation about a second pivot axis P2 extending laterally and perpendicular with the first pivot axis PI. The second pivot arm 68 is coupled with the frame 28 of the spring assembly 20 via the second adjustable joint 80 and via a third pivot joint 88 for rotation about a third pivot axis P3 extending laterally and parallel with the second pivot axis P2. Each of the pivot joints 84-88 has a single rotational degree of freedom, and none of the pivot joints 84-88 has a rotational degree of freedom about a vertical axis.

The illustrated coupling 22 includes an arcuate guide 90 formed in the second pivot arm 68 that can receive a pin or other guide follower extending from the second adjustable joint 80. Ends of the arcuate guide 90 can define positive stops placing limits on the amount of rotation of the coupling 22 relative to the spring assembly 20.

An illustrative method of fitting the ankle foot orthosis 10 to a person may thus include a adjusting the equilibrium angle a of the orthosis to match the stance angle of the person. The method may include unlocking an adjustable joint (e.g., adjustable joint 64) coupling the spring assembly 20 with the leg stay 18, rotating the spring assembly 20 about the pivot joint 16 until the cam follower 50 is at an equilibrium position along the cam profile 46, and locking the adjustable joint at that position. At least the rotation of the spring assembly 20 and the locking of the adjustable joint 64 are performed while the person is wearing the orthosis 10 and is in a standing position. The method may alternatively or additionally include adjusting a spring reaction force applied to the leg of the person wearing the orthosis during dorsiflexion and plantarflexion. The reaction force may be adjusted by unlocking an adjustable joint (e.g., adjustable joint 80) coupling the spring assembly 20 with the leg stay 18, vertically adjusting the leg stay 18 relative to the spring assembly, and locking the adjustable joint at the desired location of the leg stay. This adjustment may be performed while the orthosis is being worn or when not being worn.

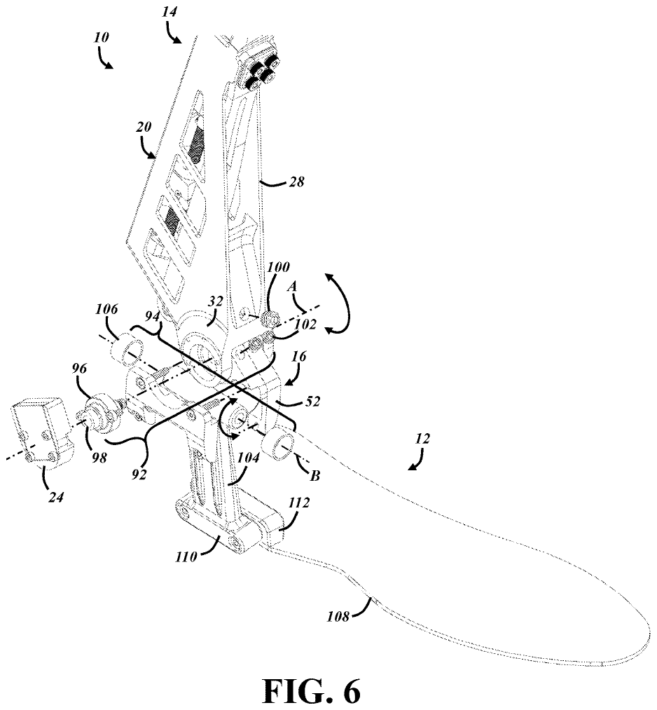

FIG. 6 is a perspective view of a lower portion of the orthosis 10 of FIG. 1 with the pivot joint 16 in exploded view. As noted above, the pivot joint 16 couples the foot portion 12 with the leg portion 14 of the orthosis 10 for relative movement about at least one pivot axis. In the illustrated example, the pivot joint 16 has two rotational degrees of freedom including a first degree of freedom about the first pivot axis A and a second degree of freedom about the second pivot axis B. The pivot joint 16 includes an ankle pitch joint 92 permitting dorsiflexion and plantarflexion about the first axis A, and an ankle roll joint 94 permitting inversion and eversion about the second axis B.

The body 52 of the pivot joint 16 and the lower end 32 of the frame 28 each form a portion of the ankle pitch joint 92 with a bushing or bearing assembly 96 at their interface. In this example, the bearing assembly 96 has an inner race pressed onto a shoulder screw axle affixed to the joint body 52 and an outer race pressed into a bore of the frame 28. As illustrated in FIG. 6, the pivot joint 16 may house a magnet 98 or other element detectable by the angle sensor 24 along the first pivot axis A. The orthosis 10 may additionally include adjustable stops 100, 102 to limit and adjust the range of dorsiflexion and plantarflexion. In this case, the plantarflexion adjuster 100 is a set screw extending through a portion of the frame 28, where the inner end of the adjuster acts as a mechanical stop in plantarflexion by limiting the amount of rotation of the joint body 52 in that direction. The illustrated dorsiflexion adjusters 102 are also set screws extending from a face of the joint body 52, where the outer ends of the adjusters act as mechanical stops in dorsiflexion by limiting the amount of rotation of the joint body 52 in that direction.

The body 52 of the pivot joint 16 and a pivot arm 104 of the foot portion 12 each form a portion of the ankle roll joint 94 with a bearing assembly or bushings 106 at their interface. In this example, the roll joint 94 includes bushings 106 on a cylindrical top portion of the pivot arm 104. As illustrated in FIG. 6, a portion of the joint body 52 may be removable for access to and/or disassembly of the roll joint 94 without disassembly of the ankle pitch joint 92. This accessibility may also enable a replaceable pivot arm 104. For example, the illustrated pivot arm 104, which is symmetric with respect to a coronal (x-z) plane, can be replaced with a different pivot arm in which the lower end 110 of the pivot arm 104 is anteriorly or posteriorly offset with respect to the axis A of the ankle pitch joint 92 to better align the pitch joint with the user's ankle joint. Adjustable stops similar to those associated with the ankle pitch joint 92 may be employed to adjustably limit the amount of eversion and inversion about the ankle roll joint 94.

In some embodiments, the ankle roll joint 94 is lockable at a plurality of angular positions about the second pivot axis B. The locking position may be infinitely adjustable (i.e., analog) within the available range of angular motion about the pivot axis B. In one example, set screws are included that pass through a portion of the joint body 52 and can be tightened onto the rotatable portion of the pivot arm 104 to lock the roll joint 94. The locking roll joint feature is provided for users who require stability along the inversion/eversion axis B, e.g., for pathological reasons. In one embodiment, the roll joint 94 has an available range of motion of 145 degrees (±72.5 degrees in each direction) and can be locked at any continuous position within that range. The total range of motion may itself be adjustable. For example, the available range of motion may be reduced in some embodiments to 85 degrees (+42.5 degrees in each direction), such as via a set screw that provides a mechanical stop for the pivot arm 104 with respect to the joint body 52. And the roll joint may be lockable within that reduced range.

The foot portion 12 of the orthosis 10 includes a foot plate 108 and the pivot arm 104 that forms part of the ankle roll joint 94. The end 110 of the pivot arm 104 opposite the pivot joint end is rigidly attached to the foot plate 108, in this case via a mounting block 112. During dorsiflexion and plantarflexion, the entire foot portion 12 and the joint body 52 rotate about the first pivot axis A with respect to the spring assembly 20. During eversion and inversion, the entire foot portion 12 rotates about the second pivot axis B with respect to the joint body 52 and the spring assembly 20. In some embodiments, the orthosis 10 can be configured for the right leg or for the left leg of a user using all, or nearly all, of the same components. For example, the orthosis 10 of FIG. 1 is configured for use on the right leg of a user. But, because most of the components are symmetric about a sagittal plane or rotationally symmetric about an axis, the configuration of FIG. 1 can be easily changed to a left-legged configuration. In particular, the second adjustable joint 80 of the coupling 22 can be unlocked and removed from the spring assembly 20, and the foot plate 108 can be removed from the end 110 of the pivot arm 104 of the foot portion 12. Because the coupling 22, including the second adjustable joint 80, is rotationally symmetric about its first pivot axis P1, the coupling 22 can then be rotated 180° about the first pivot joint PI and recoupled with the end 30 of the frame 28 and spring assembly 20 such that the pivot joint 16, spring assembly 20, and pivot arm 104 of the foot portion 12 will be lateral to or outboard of a user's left leg when the leg stay 18 is affixed to the left leg. A different foot plate 108 configured for a left foot can be attached at the end 110 of the pivot arm 104, and the angle sensor 24 can be moved to the opposite side of the pivot joint 16 to complete the transformation.

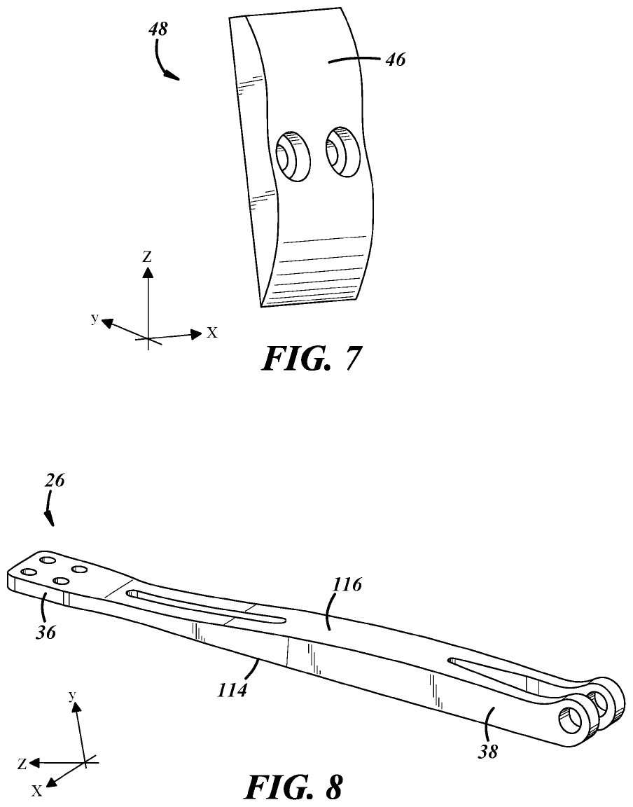

In some cases, where the foot plate 108 is (at least initially) flat, a single foot plate can be inverted for use as a right foot plate or a left foot plate with the mounting block 112 being attachable to the foot plate 108 in either orientation by rotating the mounting block about a vertical axis, thus making the orthosis truly universal in that in can be fitted to either the right leg or the left leg of any person. Moreover, the cam 48 may be made easily replaceable with a different cam having a differently shaped cam profile, making the orthosis 10 adaptable for a range of people requiring a range of different torque-angle profiles depending, for example, on their weight and/or their particular impaired ankle function. An example of a removable and replaceable cam 48 with mounting holes is illustrated in FIG. 7. As oriented in FIG. 7, the upper portion of the cam profile 46 corresponds to dorsiflexion (positive restorative torque and angle in FIG. 4) and the lower portion of the cam profile corresponds to plantarflexion (negative restorative torque and angle in FIG. 4).

While not always preferred due to its usually more expensive material, the spring 26 is also easily replaceable with a spring having a different stiffness profile. FIG. 8 illustrates an example of a removable and replaceable spring 26 with mounting holes for attachment to the frame 28 at its first end 36 and mounting holes for the cam follower 50 and its second end 38. In this example from FIGS. 1 and 6, the posterior side 114 of the spring 26 along which the spring support 34 moves is planar, while the anterior side 116 is contoured and is another portion of the spring-cam system that can be tailored to achieve the desirable torque-angle relationship of the orthosis 10.

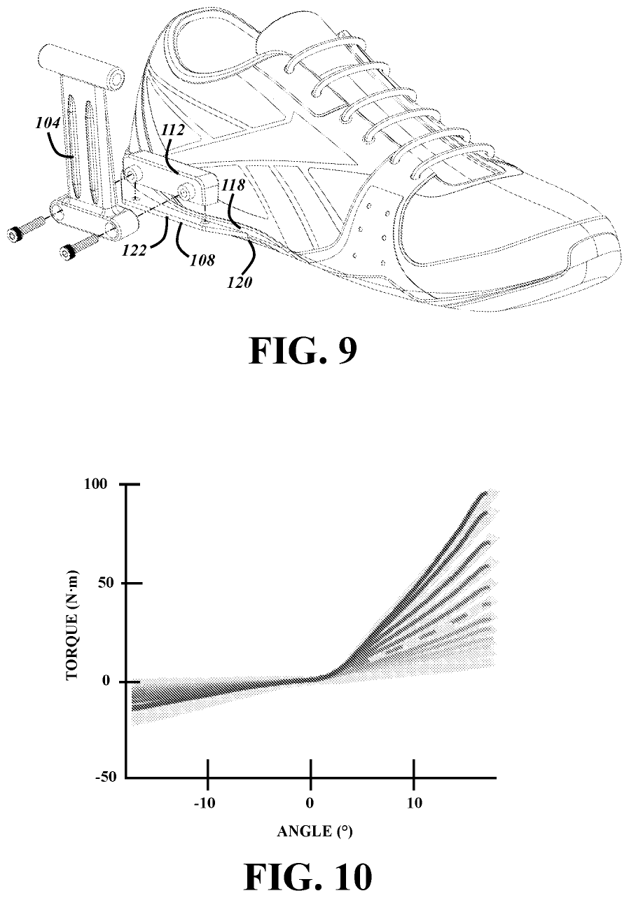

FIG. 9 illustrates integration of the foot portion 12 of the orthosis 10 into a shoe of the intended user. In this example, the foot plate 108 is interposed between layers of the sole of the shoe. One manner of achieving this integration includes splitting (e.g., cutting) the sole of the shoe into a top layer 118, which remains attached to the shoe upper, and a bottom layer 120. The layers 118, 120 may be approximately equal in thickness. The foot plate 108 can be deformed from an initial flat configuration to a contour that matches the curvature of the split layers 118, 120 and then attached to the shoe between the top and bottom layers with a structural adhesive (e.g., epoxy) or other suitable means. The mounting block 112 and/or the pivot arm 104 are affixed to a tab 122 protruding laterally from the foot plate 108. In other examples, such as that of FIG. 2, the foot plate 108 is configured as a shoe insole with a relatively thin mounting tab 122 extending vertically from the foot plate 108 lateral to the user's foot for attachment to the pivot arm 104 of the joint 16. As oriented in FIG. 7, the upper portion of the cam profile 46 corresponds to dorsiflexion (positive restorative torque and angle in FIG. 4) and the lower portion of the cam profile corresponds to plantarflexion (negative restorative torque and angle in FIG. 4). working prototype orthosis consistent with the above description has been constructed and tested as a quasi-passive ankle-foot orthosis in which a titanium spring stores and releases gravitational energy during a gait cycle. The stiffness of the spring is varied via a motorized simple support that moves toward and away from the free end of the cantilever. The cam profile governs the shape of the torque-angle function and moving the spring support makes this function more stiff or less stiff. The spring support is moved by a small motor (67 g) during unloaded leg swing, and the average joint stiffness can be adjusted within a range from 43 N·m/rad to 330 N·m/rad in dorsiflexion and within a range from 8 N·m/rad to 51 N·m/rad in plantarflexion. The orthosis is compatible with any size shoe through use of interchangeable aluminum footplates, has an adjustable range-of-movement up to 50° in both dorsiflexion and plantarflexion, and weighs approximately 1 kilogram.

The optimal torque-angle function for a passive orthosis is unknown, so the mechanics tested with the prototype orthosis (illustrated in FIG. 4) are meant to be a starting point for future exploration. The adjustable stiffness range and shape of the primary curve were influenced by able-bodied data, mechanical limits, clinical considerations, and other studies. The spring cannot replicate the net-positive mechanical work performed by a human ankle, but the stance phase of able-bodied walking is largely passive prior to push-off. The primary curve was designed to mimic the concave-up shape of able-bodied torque-angle data during controlled plantarflexion (heel-strike to foot flat) and controlled dorsiflexion (mid-stance) to imitate the quasi-stiffness and impedance of an ankle during stance. The equilibrium angle of the prototype orthosis is at 2° dorsiflexion, as in FIGS. 3-4, to facilitate foot clearance during leg swing without back-driving the transmission. The stiffness is leveled at 18° dorsiflexion to prevent high loads. The radius of the cam follower also places constraints on the second derivative of the torque-angle function and can invoke unrealizable (self-intersecting) cam geometries if it is too large.

A rotary dynamometer was used to experimentally verify the achievable ankle mechanics of the prototype. The dynamometer included a frame mounted motor and a 6-axis load cell on the motor output. The orthosis was rigidly mounted to the dynamometer at the leg stay, the ankle pitch joint was aligned with the rotational axis of the dynamometer using a concentric laser, and the foot plate was secured to an actuated rotating platform. Eleven stiffness conditions were tested, starting at the least stiff configuration (0%) and increasing in ten percent intervals until reaching the stiffest configuration (100%). At each condition the machine rotated the footplate ±18° in both dorsiflexion and plantarflexion. Ankle pitch joint angle data was collected by both the dynamometer and the on-board encoder at 100 Hz, and torque data was collected by the dynamometer's load cell at 1 KHz.

The compliance of the chassis (i.e., the frame of the spring assembly and the foot portion of the orthosis) was determined by dividing the peak torques measured by the dynamometer by the difference in angle between the encoder measurements of the orthosis and the dynamometer. The chassis had a compliance of 1450 N·m/rad in dorsiflexion and 125 N·m/rad in plantar flexion, which was input into the predictive mathematical model. As illustrated in FIG. 10, the measured torque-angle functions (solid lines) closely match model predictions (shaded regions). The model predicted a range from 39 to 315 N·m/rad average dorsiflexion stiffness and from 7 to 45 N·m/rad average plantarflexion stiffness across support conditions, whereas the experimental measurements ranged from 43 to 330 N·m/rad in dorsiflexion and from 8 to 51 N·m/rad in plantarflexion. The average stiffness of the ankle pitch joint of the orthosis can be modulated almost an order of magnitude, with an average hysteresis loss of only 5% across all trials.

The experimental results are consistent with the model and verify the desired functionality of the prototype. It is noted that, in practice people have tissue compliance that is not accounted for in the dynamometer characterization. This compliance was not measured because the results would likely be similar to experimental predictions during dorsiflexion, when the mechanism loads through bone, and varies significantly from person to person during plantarflexion. Tissue compliance is more prominent during plantarflexion when the mechanism loads the tissue behind the leg. Use of a boa strap as part of the leg stay can help minimize this compliance by applying some preload to that tissue.

The orthosis can modulate its stiffness from that of flexible AFOs used by children to rigid AFOs used by adults and opens many research possibilities such as determining the “just noticeable difference” orthotic ankle stiffness, and the ideal shape of the torque-angle function for different ankle pathologies. The orthosis may be useful for contracture or Achilles rehabilitation because the stiffness and range-of-movement can be adjustable to the user's stage of recovery. As an AFO replacement, the disclosed orthosis could even prevent contracture because the ankle mechanics can be flexible near neutral angle for increased range-of-movement and stiff in late stance for body-weight support.

The above-described orthosis may also be used as a clinician tool for orthotic stiffness prescription. For instance, a clinician could outfit a patient with the orthosis and monitor the user's gait throughout a large and continuous range of stiffness values. This tool could eliminate trial and error from the stiffness prescription process, and more quickly lead to improved patient outcomes.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. An ankle-foot orthosis comprising a leg portion and a foot portion biased toward an equilibrium angle with respect to the leg portion, wherein the equilibrium angle is adjustable.

2. The orthosis of claim 1, further comprising a spring that biases the foot portion toward the equilibrium angle, wherein the spring has a variable stiffness.

3. The orthosis of claim 1, further comprising a cam and a cam follower, one of which is mounted for movement with the leg portion about a pivot joint coupling the leg portion to the foot portion and the other of which is mounted for movement with the foot portion about the pivot joint, wherein the cam and the cam follower are biased against each other when the foot portion is away from the equilibrium angle.

4. The orthosis of claim 1, further comprising a pivot joint coupling the leg portion to the foot portion, wherein the pivot joint provides two rotational degrees of freedom.

5. The orthosis of claim 1, further comprising a pivot joint coupling the leg portion to the foot portion, wherein the equilibrium angle is adjustable about the pivot joint in dorsiflexion and plantarflexion directions.

6. The orthosis of claim 1, wherein the leg portion comprises a frame and a leg stay, the frame having a first end coupled with the foot portion at a pivot joint and a second end coupled with the leg stay, wherein a position of the second end of the frame relative to the leg stay is adjustable about the pivot joint.

7. The orthosis of claim 6, further comprising a coupling having a first pivot arm and a second pivot arm, wherein:
the first pivot arm has a first end coupled with the leg stay and a second end coupled with the second pivot arm at an adjustable joint,
the second pivot arm has a first end coupled with the frame and a second end at the adjustable joint, and
the adjustable joint provides linear adjustment of the second pivot arm relative to the first pivot arm to move the frame about the pivot joint.

8. The orthosis of claim 7, wherein a position of the first end of the second pivot arm is linearly adjustable relative to the frame.

9. The orthosis of claim 7, wherein the coupling provides pivoting movement of the frame relative to the leg stay about two parallel axes.

10. An ankle-foot orthosis comprising a leg portion and a foot portion having an equilibrium angle with respect to the leg portion, wherein a biasing force is applied to the foot portion anterior to the heel of a person wearing the orthosis when the foot portion is away from the equilibrium angle.

11. The orthosis of claim 10, wherein the equilibrium angle is adjustable.

12. The orthosis of claim 10, further comprising a pivot joint coupling the leg portion to the foot portion, wherein the pivot joint provides two rotational degrees of freedom including a first degree of freedom about a first axis in dorsiflexion and plantarflexion directions, and a second degree of freedom about a second axis in inversion and eversion directions.

13. The orthosis of claim 10, wherein the leg portion further comprises a frame and a leg stay, the frame having a first end coupled with the foot portion at a pivot joint and a second end coupled with the leg stay, wherein the frame is lateral to the leg of the person wearing the orthosis.

14. The orthosis of claim 10, further comprising a cam and a cam follower, one of which is mounted for movement with the leg portion about a pivot joint coupling the leg portion to the foot portion and the other of which is mounted for movement with the foot portion about the pivot joint,
wherein the cam and the cam follower are biased against each other by a spring when the foot portion is away from the equilibrium angle,
wherein a torque about the pivot joint resulting from a biasing force of the spring is a function of the angle of the foot portion with respect to the leg portion, and
wherein the function is at least partially defined by a profile of the cam and by a stiffness of the spring.

15. The orthosis of claim 14, wherein the stiffness of the spring is variable.

16. The orthosis of claim 15, wherein the leg portion comprises the spring, a spring support, a frame, and a leg stay, wherein:
the frame has a first end coupled with the foot portion at the pivot joint and a second end coupled with the leg stay,
the spring is a cantilever having one end attached to the frame and an opposite free end that applies the biasing force, and
the spring support is a simple support for the cantilever and is movably mounted on the frame for movement between the ends of the cantilever to vary the stiffness of the spring.

17. The orthosis of claim 16, wherein movement of the spring support is motorized.

18. The orthosis of claim 16, wherein the spring support is posterior to the spring.

19. The orthosis of claim 14, wherein the cam is mounted for movement with the foot portion and the cam follower is attached to the spring such that the cam pivots about an axis of the pivot joint and the cam follower is biased against the cam by the spring.

20. The orthosis of claim 19, wherein the cam is at the pivot joint.

21. An ankle-foot orthosis comprising a leg portion and a foot portion coupled at a pivot joint having two rotational degrees of freedom.

22. The orthosis of claim 21, wherein the two degrees of freedom include a first degree of freedom about a first axis in dorsiflexion and plantarflexion directions, and a second degree of freedom about a second axis in inversion and eversion directions.

23. The orthosis of claim 22, wherein:
the pivot joint comprises a joint body,
the joint body and the foot portion pivot together about the first axis with respect to the leg portion, and
the foot portion pivots about the second axis with respect to the joint body.

24. The orthosis of claim 22, further comprising a sensor configured to determine an angle of the foot portion with respect to the leg portion about the first axis.

25. The orthosis of claim 22, wherein a range of angular motion of the foot portion with respect to the leg portion about each axis is adjustable.

26. An ankle-foot orthosis configured for use on one of: a right leg or a left leg,
wherein the orthosis is convertible for use on the other leg by replacing only a foot plate of the orthosis.

27. The orthosis of claim 26, further comprising:
a pivot arm configured for removable attachment to the foot plate at one end and forming part of a pivot joint at an opposite end;
a spring assembly forming part of the pivot joint at one end and removably attached to a coupling at an opposite end; and
a leg stay attached to the coupling,
wherein, when converted for use on the other leg, an orientation of the pivot arm, the pivot joint, and the spring assembly does not change, and the coupling is rotated 180 degrees about an axis extending in the anterior and posterior directions.

28. The orthosis of claim 27, further comprising an angle sensor mounted on a first side of the pivot joint, wherein the sensor is mounted on an opposite second side of the pivot joint when converted for use on the other leg.

29. A method of fitting an ankle foot orthosis to a person, the method comprising the step of adjusting an equilibrium angle of the orthosis to match a stance angle of the person.

30. The method of claim 29, wherein the orthosis includes a cam having a cam profile, a cam follower, and a spring assembly configured to bias the cam and cam follower toward each other when the orthosis is not at the equilibrium angle, the method comprising the following steps:
(a) unlocking an adjustable joint coupling the spring assembly with a leg stay of the orthosis;
(b) rotating the spring assembly about a pivot joint of the orthosis until the cam follower is at an equilibrium point along the cam profile; and
(c) locking the adjustable joint after step (b),
wherein steps (b) and (c) are performed while the person is wearing the orthosis and in a standing position.

31. The method of claim 30, wherein the adjustable joint is a linearly adjustable joint.

32. The method of claim 29, further comprising the step of adjusting a reaction force applied to the leg of the person wearing the orthosis during dorsiflexion and plantarflexion.

33. The method of claim 29, wherein the method comprises the following steps:
(a) unlocking an adjustable joint coupling a spring assembly with a leg stay of the orthosis;
(b) vertically adjusting the leg stay relative to the spring assembly; and
(c) locking the adjustable joint after step (b).

34. An ankle-foot orthosis comprising a leg portion and a foot portion biased toward an equilibrium angle about a pivot joint, wherein the pivot joint has a non-linear torque-angle function.

35. The orthosis of claim 34, further comprising a spring that biases the foot portion toward the equilibrium angle, wherein the spring has a variable stiffness.

36. The orthosis of claim 34, further comprising a cam and a cam follower, one of which is mounted for movement with the leg portion about a pivot joint coupling the leg portion to the foot portion and the other of which is mounted for movement with the foot portion about the pivot joint, wherein the cam and the cam follower are biased against each other when the foot portion is away from the equilibrium angle and the non-linear torque-angle function is defined at least in part by a cam profile of the cam.

37. The orthosis of claim 34, wherein the equilibrium angle is adjustable.

38. The orthosis of claim 34, wherein the pivot joint has two rotational degrees of freedom.
